(19) United States
(12) Patent Application Publication
(10) Pub. No.: US 2016/0166173 A9
(43) Pub. Date: Jun. 16, 2016

(54) Device For The Detection Of Metallic Surgical Articles And Harmonic And RFID Tagging Markers

(71) Applicant: Integrated Medical Technologies, Inc., Bloomington, IL (US)

(72) Inventors: William B. Smith, Bloomington, IL (US); Nelson S. Slavik, Niles, MI (US); Michael H. Linse, Corvallis, OR (US)

(21) Appl. No.: 14/082,114

(22) Filed: Nov. 16, 2013

Related U.S. Application Data
(60) Provisional application No. 61/727,348, filed on Nov. 16, 2012.

(51) Int. Cl. A61B 5/06; A61B 8/08; A61G 13/10; A61B 19/00

(52) U.S. Cl. CPC A61B 5/064; A61B 19/5244; A61B 8/0841; A61B 19/2203; A61B 19/5212; A61G 13/10; A61B 19/44; A61B 19/54; A61B 2019/5255; A61B 2019/5263; A61B 2019/448; A61B 2019/5425; A61B 2019/5437

(57) ABSTRACT

The present invention provides a method of detection of metallic items and/or metallic surgical instruments or articles by a detection device in the event that such articles have been retained in a patient after a surgical procedure. More specifically, the invention relates to a detection system that uses (1) a metal detector or (2) a detector for harmonic-generating amorphous or non-amorphous metallic tags together with three-dimensional spatial position measurement to determine if a foreign metallic article has been retained in the body and if so, to determine the article's location. The invention scans the patient pre- and post-operatively, evaluating three-dimensional differences in electronic response to account for other nearby metallic objects not related to the retained metallic article, thereby determining by inference whether the post-operative electronic response results from presence of a retained article.

### RELATED APPLICATIONS

This application is a continuation-in-part of U.S. application Ser. No. 61/727,348, filed on Nov. 16, 2012, the entire content of which is incorporated by reference herein.

### BACKGROUND OF THE INVENTION

The invention generally relates to a device for detection of metallic items or tagged non-metallic items that are erroneously retained in the patient after a surgical procedure. Medical practitioners occasionally make the error of leaving an object within the surgical patient. Such an event can cause devastating injury or death to the patient. Various approaches have been taken with the goal of solution to the retained-object threat. Counting the surgical equipment before and after its application within the patient is a common preventive measure. This is not completely effective.

Several kinds of electronically detectable tags, such as RFID tags, have been developed. These tags are applied, primarily to surgical sponges and pads, and an electronic detection instrument is used to determine that no tag responses arise when the detector is used to scan the surgical area. Application of tags to surgical tools and instruments is problematic due to the challenge of acceptable attachment to the varied shapes and sizes of these items. The complications of tag application to surgical instruments commonly result in the decision not to tag these kinds of equipment. Accounting for non-tagged equipment is occasionally erroneous.

X-ray imaging can be used for detection of retained instruments or even surgical sponges, but this detection method produces radiation exposure, and it is most often applied only after patient distress gives rise to suspicion that a retained object is present within the patient.

Metal detectors have been proposed in the past for use in discovery of retained metallic items that are left within the patient. The successful use of metal detection for this purpose is severely hampered by the fact that the surgical environment includes metal objects, such as structural components of the operating table, for example, that are confounding to the successful use of hand-held metal detection devices. Metal detectors respond to changes in a magnetic field that occur due to properties of the metal targets that are present. In general, these properties include magnetic permeability and/or electrical conductivity, as well as size, shape, and orientation. Target objects that have been retained within the patient may be large or small, and orientation might or might not favor detection In this practice, the response of the metal detection device is complex when it is applied in such an environment, too much so for reliable use of a hand-maneuvered metal detector and interpretation of its indications by a human operator for determination that a retained metallic object is or is not present within the patient. The use of hand-maneuvered detectors is dependent upon diligence on the part of the operator in that the operator must perform a scan that covers the area of interest both completely and in a sufficiently fine search pattern to avoid gaps in coverage. The operator can fail to achieve this goal.

### SUMMARY OF THE INVENTION

The present invention provides an electromagnetic metal detection system for detection of retained metallic surgical equipment which applies new methods to address the complexity of metal detection information in the surgical environment.

We provide a metal detector or a detector for harmonic-generating amorphous or non-amorphous metallic tags that are used to scan the surgical area prior to the surgical procedure, and to scan the surgical area again following the surgical procedure. Fixed metallic items that are present in the surgical area result in detection signals in both scans, while a retained metallic object or tagged non-metallic results in a detection signal for the retained object that is present only in the post-surgical scan, but which is combined with other signals from the fixed objects that are present. Electromagnetic metal detectors can be implemented in several ways. They share the features that are important in this invention: That they produce a time-varying excitation field and that they measure disturbance of this field to observe the effect of the presence of metal within the excitation field. The excitation field is typically produced by current in some kind of loop, This excitation current can be applied as a pulse or as a continuous alternating current (AC) signal. Detection is achieved in the pulse-mode system by observation of the amplitude and damping of the detected signal. Detection is achieved in the AC case by observation of the amplitude and phase of the detected signal. In addition to its desired responses to metal objects, metal detection systems are sensitive to the distance of the detection sensor to the target, and they are sensitive to orientation of the excitation-detection loops. Thus, in addition to the process of accounting for detection signals in scans prior to and after the surgery, the present invention also accounts for the orientation and path through which the detector travels during the scans.

Computation to subtract the data of the first scan from the data of the second scan, and to account for the paths and orientations of the scans, results in a set of difference data in which the detection results for objects that were present in both scans are suppressed, thus enhancing the visibility of detection signals that result from the presence of metallic objects that were left in the patient.

### BRIEF DESCRIPTION OF THE DRAWINGS

FIG. 1 is a side view of a patient on a typical operating table.

FIG. 2 is an overhead view of a patient on a typical operating table.

FIG. 3 is an overhead view of the patient on the operating table, showing typical path of the detector in pre-surgery scan, and graphs of typical detection signal magnitudes with no metallic object retained in the patient.

FIG. 4 is an overhead view of the patient on the operating table, showing typical path of the detector in post-surgery scan, and graphs of typical detection signal magnitudes with presence of a metallic object retained in the patient.

FIG. 5 is a side view of the patient on the operating table, showing typical elevation of the detector's loop path above the patient.

FIG. 6 is an overhead view of the patient on the operating table, showing graph of the magnitude of computed results of subtraction of the pre-surgery detection signals from the post-surgery detection signals.

FIG. 7 is an overhead view of the patient on the operating table, showing detector path achieved by use of a robotic positioner, also showing graph of the magnitude of computed results of subtraction of the pre-surgery detection signals from the post-surgery detection signals.

FIG. 8 is a side view of the patient on the operating table, showing detector mounted on a robotic positioner.

FIG. 9 Is a side view showing installation of robotic system.

FIG. 10 is an overhead view of patient and table-mounted robotic system

### DETAILED DESCRIPTION OF THE PREFERRED EMBODIMENT

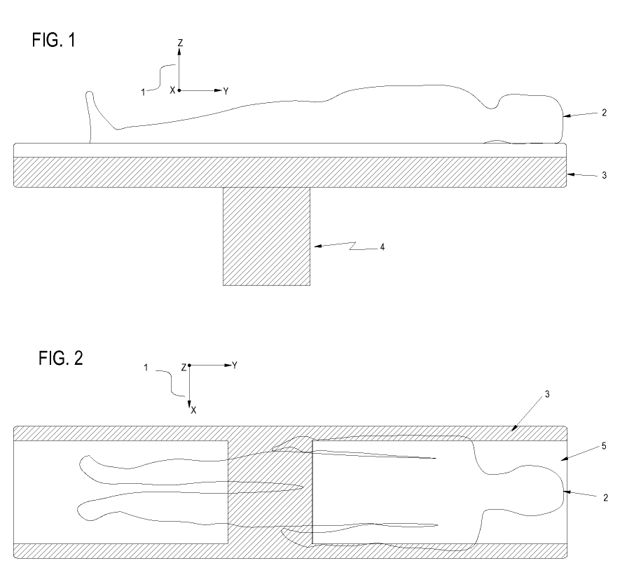

FIG. 1 depicts a side or profile view of a surgical patient 2 upon a padded operating table 3. The operating table is supported upon a pedestal 4. X, Y, and Z axes for reference in this and subsequent figures are shown at 1 in this figure. The hatched areas shown at pedestal 3 and table 4 are commonly comprised of metallic structural components, or a composite of metallic and non-metallic components. Operating tables are commonly adjustable for height and incline by use of mechanical features that are parts of the pedestal and of the structural frame of the table.

FIG. 2 is an overhead view of the patient 2 and table as shown in FIG. 1. Note that the table includes area 3 which is a mix of metallic and non-metallic components, and area 5 which is non-metallic to provide a radiolucent area that permits radiography through the table.

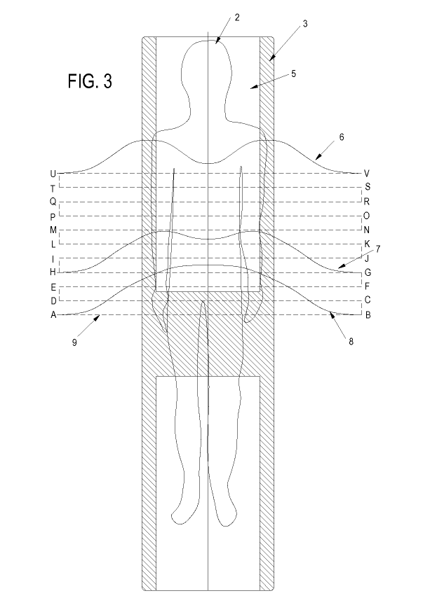

FIG. 3 is an overhead view of the patient 2 and table. Dashed line 9 shows a typical path for passage of a metal detector's sensor over the patient to scan the surgical area prior to performance of the surgical procedure. The path 9 is composed of line segments AB, BC, CD, and so forth, ending at point V. Graph 6 depicts the magnitude of the metal detection signal along path 9's segment between points U and V. Note that graph 6 shows increased signal level in the areas in which the detector passes over metallic features 3 of the operating table. In similar fashion, graph 7 depicts magnitude of the metal detection signal along segment GH of path 9, also showing increased signal level in the areas in which the detector passes over metallic features 3 of the operating table. Graph 8 depicts magnitude of the metal detection signal along segment AB of path 9, showing the effect of the metallic area 3 of the operating table in this region.

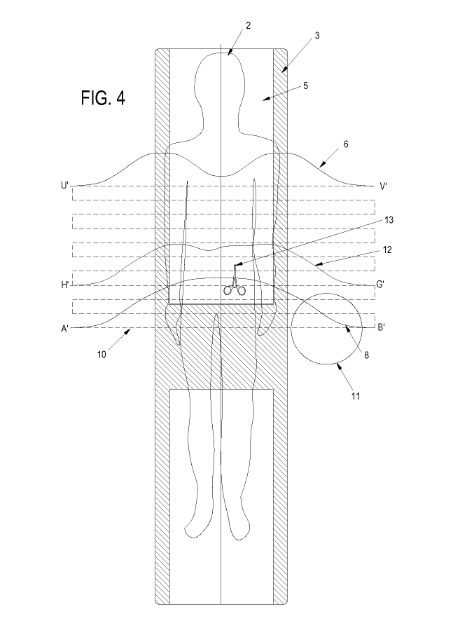

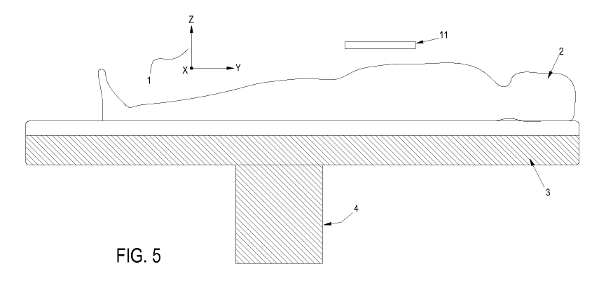

FIG. 4 is an overhead view of the patient 2 and table. Dashed line 10 shows a typical path for passage of a metal detector's sensor over the patient to scan the surgical area after the surgical procedure. The path 10 is composed of line segments identified by A′ to V′, a path which closely matches the path 9 of FIG. 3. The circle 11 shows typical proportion of a metal detection assembly containing excitation and detection loop or loops. The detector loop size is typically chosen with radius equal to the distance at which detection targets might be found, although reduction from that radial size still provides acceptable performance. Note that path 9 and path 10 show the loci of positions of the center of the detection loop. Detection signal graphs 6 and 8 are unchanged from the pre-surgical scan of FIG. 3, but graph 12, along the path G′H′ that produced graph 7 of FIG. 3 along path GH, is changed due to the presence of a retained foreign body, forgotten surgical tool 13. The differences between the signal magnitudes of graph 7 and graph 9 are generally not perceived by a worker who performs these scans by use of a hand-held detector, because the pre-surgical scan is usually separated in time by an interval of time ranging from several minutes to several hours, and because the signals do not differ to a large extent. In addition, signal levels can be affected as much by distance and orientation of the detector as by presence of retained foreign body 13. Acceptable duplication of the pre-surgical scan should achieve about +/−0.1 inch position matching.

The metal detector in this system is a so-called induction balance or pulse-induction metal detector having design consistent with the art of metal detection. The unique feature of the metal detector in the present invention is means to concurrently store data for position of the detection device together with storage of the metal detection measurements that are made during application of the detector to scan patient 2 before and after conduct of the surgical procedure. Means are also provided for computation of the difference between measurement data of the two scans to yield a resulting set of data in which the signals that were present at the same or similar amplitude in both scans are suppressed, thus enhancing the signals from metallic retained foreign bodies, if any such foreign bodies are present. Means are also provided for display of the detection data with reference to the dimensions of the area that was scanned.

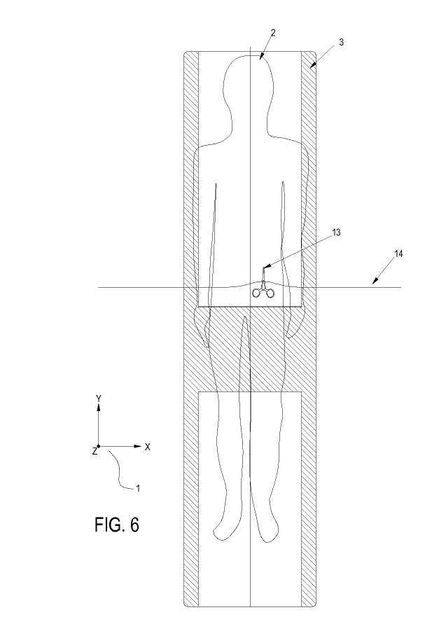

FIG. 5 is a side or profile view of surgical patient 2, with metal detector loop 11 shown in typical elevation above patient 2. Optimal operation of the detector will maintain the plane of loop 11 parallel to the plane of the operating table 3. The elevation of post-surgical scan path 10 of FIG. 4 should match the elevation of path 9 of FIG. 3.

FIG. 6 shows the metal-detection result that is obtained by combination of the data of the pre-surgical scan with the data of the post-surgical scan so as to obtain the difference of the two data sets. FIG. 6 is an overhead view of the patient 2 and table 3. Retained foreign body 13 is shown, and graph 14 depicts the difference between magnitudes of the metal detection data of the post-surgical scan and the pre-surgical scan. Although the signal resulting from retained foreign body 13 may be relatively smaller than the signals resulting from fixed metallic objects in the surgical area, the method of the present invention achieves suppression of the signals from objects that are present in both scans, leaving the signal resulting from the presence of retained foreign body 13 enhanced for easier recognition by the operator of this detection system.

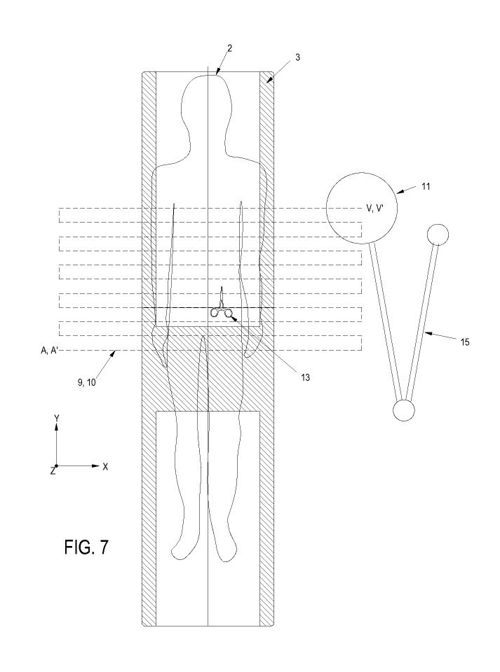

FIG. 7 shows the mechanism that is used to perform scanning of path 9 and path 10 with acceptable matching of spatial positions. Metal detection loop 11 is carried upon robotic positioner 15. Robotic positioner 15 provides positioning means to move the metal detection loop 11 along pre-surgical detection scan path 9 and post-surgical detection scan path 10 with close matching of these paths. In this depiction the robotic positioner can be retracted clear of the surgical area to leave clear access around the operating table 3. Alternate placement of the robotic positioner 15 would use a mounting fixture on the operating table 3 or the position on the floor of the base of robotic positioner 15 would be marked to provide means to facilitate the removal of the robotic positioner 15 from and restoration of the robotic positioner 15 to this marked position. The goal in application of robotic positioner 15 is acceptable match of the positions of paths 9 and 10.

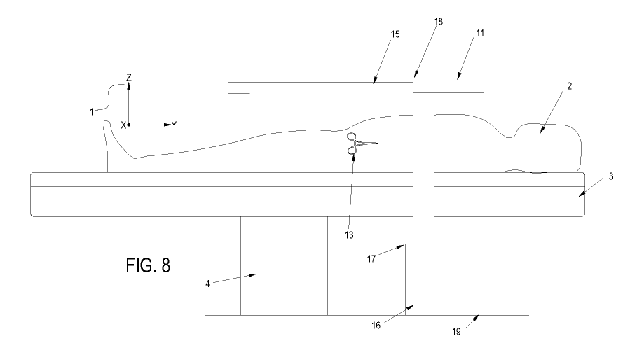

FIG. 8 is a side view of the system shown in the overhead view given in FIG. 7. The robotic positioner 15 is shown to be combined with the mounting base 16, which stands on floor 19. Elevating means and rotating means at 17 are combined into base 16. Thus the robotic positioning system can control the location of detection loop 11 in the X, Y, and Z axes. It can be desirable to apply the detection scanner in a setting in which the operating table 3 is inclined, in which case articulation means 18 would be included in the attachment of detection loop 11 for the purpose of orientation of detection loop 11 so as to make the plane of detection loop 11 parallel to the plane of operating table 3.

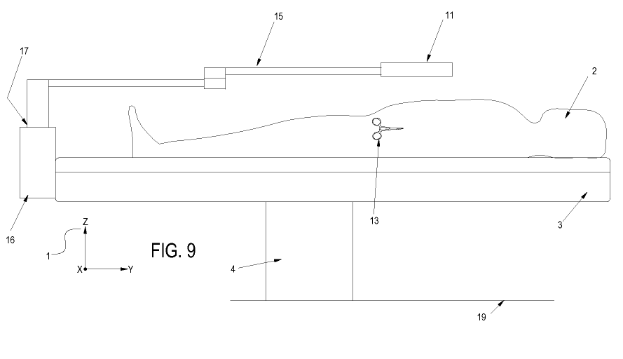

FIG. 9 is a side view showing installation of robotic system 15, 16, 17 onto the operating table. This results in parallel orientation of the planes of the detection loop 11 and the operating table 3, without need for additional ability to robotically control articulation of the detection loop 15.

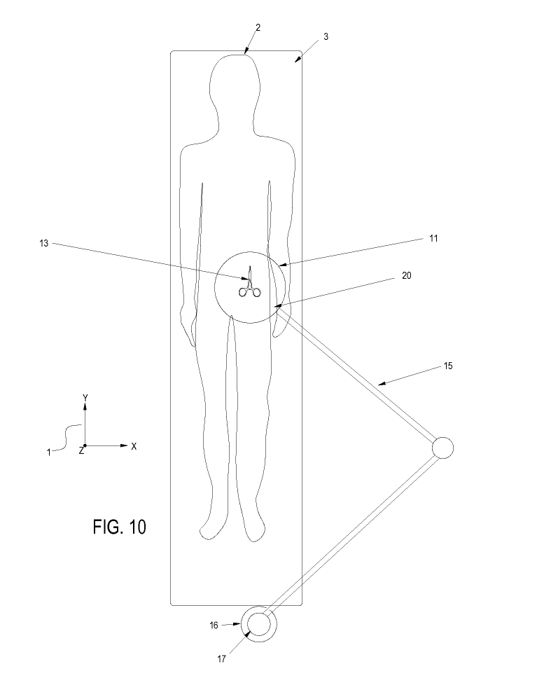

FIG. 10 is an overhead view of patient 2, table 3, robotic system 15, 16, 17, and detection loop 11. When pre-surgical and post-surgical scans are completed, and when the difference between the data sets from these respective scans is computed, the robotic system uses projection means 20, which are included in the detection loop assembly, to provide an indication of the location of the area in which detection data results indicate suspicion of the existence of a retained foreign body 13, said projection being presented directly upon the body of patient 2. Said projection means can provide indication by illumination of the suspect area, or by projection of a graph of the differential detection data that was computed by analysis of the pre-surgical scan and the post-surgical scan, or both.

We have shown and described the preferred embodiment of this invention. It is to be understood that the invention is not limited to the form of the preferred embodiment that is presented herein, and that it may be embodied in other forms within the scope of the following claims.

We claim:

1. An improvement to detection systems used to discover retained foreign metallic objects in surgery, comprising
a) A metal detector, and
b) A system to perform accurate measurement of the spatial position of the metal detector as it is used to scan over the surgical patient before and after surgery, and
c) Means for recording the position and metal detection data for the scans made before and after surgery, and
d) Means for combining the recorded metal detection data to compute the difference between the detection data taken after surgery and the data taken before surgery, and
e) Means for display of results of the computations of (d) as an image projected onto the scanned area of the patient to display areas where presence of a retained foreign metallic body is suspected as a result of the detection scans of (b).
f) Means for display of results of the computations of (d) as an image of the detection map upon a printer or graphic display panel.

2. The improved detection system of claim 1 in which the system to perform accurate measurement of the spatial position of the metal detector is a robotic positioner having automated path control of the robotic positioner and computer-measurable position sensors included as a part of the robotic positioner.

3. The improved detection system of claim 1 in which the metal detector is hand-held and the system to perform accurate measurement of the spatial position of the metal detector is a system such as video position measurement, ultrasonic position measurement, laser position measurement, or inertial navigation position sensing measurement system, or a combination of spatial position measurement systems in use to measure position and orientation of the metal detector during scanning.

4. The improved detection system of claim 1 in which the robotic positioning system is attached to the operating table.

5. The improved detection system of claim 1 in which the robotic positioning system is not attached to the operating table.

6. The improved detection system of claim 1 in which the image projected onto the scanned area of the patient to display areas where presence of a retained foreign metallic body is suspected as a result of the detection scans is simple illumination of the suspect area.

7. The improved detection system of claim 1 in which the image projected onto the scanned area of the patient to display areas where presence of a retained foreign metallic body is suspected as a result of the detection scans is a graph showing magnitude of the metal detection data.

8. An improvement to detection systems used to discover retained foreign objects in surgery using harmonic-generating tags, comprising
a) A detector for amorphous-metal harmonic-generating tags, and
b) A system to perform accurate measurement of the spatial position of the metal detector as it is used to scan over the surgical patient after surgery, and
c) Means for recording the position and metal detection data for the scan after surgery, and
d) Means for computing location of apparent tag detection from recorded detection data taken after surgery, and
e) Means for display of results of the computations of (d) as an image projected onto the scanned area of the patient to display areas where presence of a retained foreign metallic body is suspected as a result of the detection scans of (b).
f) Means for display of results of the computations of (d) as an image of the detection map upon a printer or graphic display panel.

9. The improved detection system of claim 8 in which the system to perform accurate measurement of the spatial position of the tag detector is a robotic positioner having automated path control of the robotic positioner and computer-measurable position sensors included as a part of the robotic positioner.

10. The improved detection system of claim 8 in which the metal detector is hand-held and the system to perform accurate measurement of the spatial position of the metal detector is a system such as video position measurement, ultrasonic position measurement, laser position measurement, or inertial navigation position sensing measurement system, or a combination of spatial position measurement systems in use to measure position and orientation of the metal detector during scanning.

11. The improved detection system of claim 8 in which the robotic positioning system is attached to the operating table.

12. The improved detection system of claim 8 in which the robotic positioning system is not attached to the operating table.

13. The improved detection system of claim 8 in which the image projected onto the scanned area of the patient to display areas where presence of a retained foreign tagged body is suspected as a result of the detection scan is simple illumination of the suspect area.

14. The improved detection system of claim 8 in which the image projected onto the scanned area of the patient to display areas where presence of a retained foreign tagged body is suspected as a result of the detection scan is a graph showing magnitude of the metal detection data.

15. The improved detection system of claim 8 in which the detection tag is an amorphous-metal harmonic-generating tag.

16. The improved detection system of claim 8 in which the detection tag is a crystalline-metal harmonic-generating tag.
